(19) United States
(12) Patent Application Publication
(10) Pub. No.: US 2024/0156713 A1
(43) Pub. Date: May 16, 2024

(54) Concealer sunscreen facial cream and its preparation method

(71) Applicant: Shenzhen Okary Biotech Co.,Ltd., Shenzhen (CN)

(72) Inventor: ZEDA CHEN, Shenzhen (CN)

(21) Appl. No.: 17/985,929

(22) Filed: Nov. 14, 2022

(51) Int. Cl. A61K 8/894; A61K 8/19; A61K 8/23; A61K 8/26; A61K 8/34; A61K 8/36; A61K 8/37; A61K 8/44; A61K 8/891; A61K 8/92; A61Q 1/02; A61Q 17/04

(52) U.S. Cl. CPC A61K 8/894; A61K 8/19; A61K 8/23; A61K 8/26; A61K 8/345; A61K 8/361; A61K 8/375; A61K 8/44; A61K 8/891; A61K 8/92; A61Q 1/02; A61Q 17/04; A61K 2800/524

(57) ABSTRACT

The present invention provides a concealer sunscreen cream and its preparation method; including the following weight percentages of each component: polydimethylsiloxane PEG-10/15 cross-linked polymer/polydimethylsiloxane 3%, KF6038 1.5%, polyglyceryl-3 diisostearate 0.3%, KSP-300 1.0%, KSP-100 1.5%, bismuth chloride 1.8%, coconut oil-caprylic/capric acid esters 2%, ethylhexyl hydroxy stearate 2%, KP-545 2%, isonononyl isononanoate 2%, silicone oil 1.5%, Ve 0.5%, synthetic wax SW3254 2%, synthetic wax M80 4%, D5 10%, low viscosity silicone oil 1%, silicone bullet 4%, glycerin 5%, butylene glycol 4%, glycerin polyether-26 5%, magnesium sulfate 0.7%, Pro-Xylane 5%, water 39.15%, aluminum octyl succinate 0.5%, phenoxyethanol 0.3%, chlorphenesin 0.15%, fragrance 0.1%. Ve is added to the components of the present invention, which makes the product has the effect of whitening and moisturizing and delaying skin aging; synthetic wax SW3254 and synthetic wax M80 are added to the components of the present invention, which improves the emulsification of the whole system; D5 is added to provide a refreshing greasy skin feeling and reduce the greasy feeling.

### TECHNICAL FIELD

The present invention relates to the field of facial creams: in particular, it relates to a concealer sunscreen facial cream and its preparation method.

### BACKGROUND

The role of facial cream is mainly to conceal, adjust the skin tone, sunscreen, meticulous pores, which can create a visual effect of nude makeup. However, the existing facial creams on the market are added with preservatives such as phenoxyethanol, some people will have allergies after use, causing harm to people's skin, resulting in a poor range of application of the product, and the conditioning agents used in commercially available facial creams are generally chemical composition substances, which are more irritating to the skin compared to natural extracts.

### SUMMARY

The purpose of the present invention is to provide a concealer sunscreen facial cream and its preparation method.

The present invention is implemented by the following technical solutions:

The present invention relates to a concealer sunscreen facial cream, including the following weight percentages of each component:

| | |
|---|---|
| polydimethylsiloxane PEG-10/15 cross-linked | 3%, |
| polymer/polydimethylsiloxane | |
| KF6038 | 1.5%, |
| polyglyceryl-3 diisostearate | 0.3%, |
| KSP-300 | 1.0%, |
| KSP-100 | 1.5%, |
| bismuth oxychloride | 1.8%, |
| coco-octanoic/capric acid | 2%, |
| ethylhexyl hydroxy stearate | 2%, |
| KP-545 | 2%, |
| isononyl isononanoate | 2%, |
| silicone oil (6 viscosity) | 1.5%, |
| Ve | 0.5%, |
| synthetic wax SW3254 | 2%, |
| synthetic wax M80 | 4%, |
| D5 | 10%, |
| low viscosity silicone oil | 1%, |
| silicone elastomer | 4%, |
| glycerin | 5%, |
| butylene glycol | 4%, |
| glycerol polyether-26 | 5%, |
| magnesium sulphate | 0.7%, |
| Pro-Xylane | 5%, |
| Water | 39.15%, |
| modified starch (aluminum octyl succinate) | 0.5%, |
| antiseptic (phenoxyethanol: 60+) | 0.3%, |
| chlorphenesin: 280 | 0.15%, |
| fragrance | 0.1%. |

The components in the concealer sunscreen facial cream covered by the present invention act as follows:

Polydimethylsiloxane PEG-10/15 cross-linked polymer/polydimethylsiloxane covered by the present invention, where the polydimethylsiloxane PEG-10/15 cross-linked polymer is a film-forming agent and a surfactant; meanwhile, the polydimethylsiloxane PEG-10/15 cross-linked polymer is not acne-causing.

KF6038 covered by the present invention, where the molecule contains a modified silicone oil with a polyoxyethylene group and an organosilicon group. It is an excellent silicone oil with W/O and W/Si emulsifier properties. It is deodorized. Side chains of grafted silicone can effectively stabilize low viscosity systems. Refreshing and stable W/Si emulsion can be made; the role of KF6038 covered by the present invention in the present invention is: sunscreen function.

The main role of polyglyceryl-3 diisostearate covered by the present invention is emulsifier with risk factor of 1, which is relatively safe and can be used with confidence and no effect on pregnant women in general.

KSP-300 and KSP-100 covered by the present invention have excellent smoothing, elasticity and grease absorption properties.

The bismuth chlorooxide covered by the present invention is generally filler, skin conditioning agent, coloring agent; but its main role in the skin care products covered by the present invention is filler with risk factor of 1, which is relatively safe and can be used with confidence and no effect on pregnant women in general, and bismuth chlorooxide is not acne-causing.

The main role of coco-alpha-octanoic acid/decanoic acid esters covered by the present invention is emollient with risk factor of 1, which is relatively safe and can be used with confidence and no effect on pregnant women in general, and coco-alpha-octanoic acid esters/decanoic acid esters are not acne-causing.

The main role of ethylhexyl hydroxystearate covered by the present invention in cosmetics and skin care products is emollient, with a risk factor of 1, which is relatively safe and can be used with confidence and no effect on pregnant women in general, ethylhexyl hydroxystearate is acne-causing, and consumers with sensitive skin should pay more attention to it: in addition, ethylhexyl hydroxystearate is a synthetic ester, which can play a softening role and be used as a skin conditioning agent and emollient in cosmetics.

KP-545 covered by the present invention is a volatile silicone oil soluble product with acrylic acid as the main chain and side linking silicone. After the volatile components evaporate, a soft skin film with high hydrophobicity, lubricity and oxygen permeability can be obtained. In addition, the dispersively of inorganic powder can be improved.

Isonononyl isononanoate covered by the present invention is an emollient, and isononyl isononanoate composition is suitable for 7 types of skin: sensitive skin, firm skin, non-pigmented skin, dry skin, wrinkled skin, tolerant skin, and pigmented skin.

Silicone oil (6 viscosity) covered by the present invention is colorless and tasteless, with no effect on the appearance and aroma of the final product; with a sense of ease in use and good unfolding; with good hydrophobicity and smoothness; a non-toxic and non-hazardous product without irritation to the skin and high safety to human body; with good gas permeability and no obstruction to skin breathing; with good chemical stability no react with other cosmetic raw materials.

Ve covered by the present invention has the effect of whitening and moisturizing, delaying skin aging.

Synthetic wax SW3254 and synthetic wax M80 covered by the present invention are mainly used in cosmetics and skin care products as emollient and emulsifying stabilizer with risk factor of 1, which are relatively safe and can be used with confidence and no effect on pregnant women in general, and the synthetic wax is not acne-causing.

D5 covered by the present invention is decamethylcyclopentasiloxane, which can provide a refreshing greasy skin feeling.

Low viscosity silicone oil covered by the present invention moisturizes the skin.

Silicone elastomer covered by the present invention can play a certain moisturizing role, which can form a layer of oil film on the skin surface to help protect the skin and is more suitable for people with dry skin type.

Glycerin covered by the present invention can form an invisible protective film on the surface of the skin to keep the skin isolated from the outside air and protect the skin from the attacks brought by the outside environment, thus playing a role in protecting the skin.

Butylene glycol covered by the present invention is mainly a moisturizing ingredient.

Butylene glycol helps the skin to retain more moisture and reduce the dryness of the skin after it is applied to the skin.

Glycerol polyether-26 covered by the present invention is a glycerol addition product with a good moisturizing and lubricating effect and some benefits for the skin, which is added to cosmetics and makeup remover products to obtain a good protective effect on the skin.

Magnesium sulphate covered by the present invention has an obvious improvement effect on various eczema, edema caused by trauma, local inflammation of skin and other symptoms.

Pro-Xylane covered by the present invention is a glycoprotein compound that can stimulate the conversion of hydroxyglucan in the skin, and the key effect is to delay aging.

Aluminum octyl succinate covered by the present invention can increase the consistency of the system, reduce the greasy feeling, and play a role in improving the skin feeling of the product.

Phenoxyethanol covered by the present invention has a certain effect on skin beauty and skin care. Skin roughness and enlarged pores are relieved to some extent after using, which is a preservative and generally has no effect on human skin. Phenoxyethanol plays a bactericidal role in cosmetics.

In addition to the above-mentioned respective roles of the components of the present invention, there are interactions with each other to finally achieve the purpose of the present invention which acts as concealer and sunscreen.

The present invention also relates to the aforementioned preparation method of the concealer sunscreen facial cream, including the following steps:
Step 1, weighing the raw materials according to the mass ratio of the main ingredients;
Step 2, the weighed raw materials are heated and dissolved, emulsified, cooled, and the finished product is obtained after the filling test is passed.

The present invention has the following advantages:
(1) The product uses phenoxyethanol as a preservative, with the effect of effectively inhibiting microorganisms in the air, which is not only non-toxic and harmless, but also achieves the effect of preservative, which is more natural and harmless and applicable to more people; the product is not a simple combination, and the scientific ratio and reasonable preparation method make it more effective, in addition, its scientific preparation method is suitable for batch production with higher production efficiency.
(2) The present invention adds KF6038 as a component, which interacts with polydimethylsiloxane PEG-10/15 cross-linked polymer/polydimethylsiloxane to finally realize the sunscreen function; KSP-300 and KSP-100 are also added to the present invention, which can realize excellent smoothness, elasticity and grease absorption; the invention also adds KP-545 to make the dispersivity of each component of the product stronger.
(3) The present invention adds Ve as a component, which interacts with other components to finally make the product has the effect of whitening and moisturizing and delaying skin aging.
(4) Synthetic wax SW3254 and synthetic wax M80 are added to the components of the present invention, which improves the emulsification of the whole system and makes each component give full play to its function; in addition, D5 is also added to provide a refreshing greasy skin feeling and reduce the greasy feeling.

### DETAILED DESCRIPTION OF EMBODIMENTS

The following is a detailed description of the present invention in conjunction with specific examples. It should be noted that the following examples are only further descriptions of the present invention, but the scope of protection of the present invention is not limited to the following examples.

### Example 1

The present example relates to a concealer sunscreen facial cream, including the following weight percentages of each component:

| | |
|---|---|
| polydimethylsiloxane PEG-10/15 cross-linked | 3%, |
| polymer/polydimethylsiloxane | |
| KF6038 | 1.5%, |
| Polyglyceryl-3 diisostearate | 0.3%, |
| KSP-300 | 1.0%, |
| KSP-100 | 1.5%, |
| Bismuth oxychloride | 1.8%, |
| Coco-octanoic/capric acid | 2%, |
| Ethylhexyl hydroxy stearate | 2%, |
| KP-545 | 2%, |
| Isononyl isononanoate | 2%, |
| Silicone oil (6 viscosity) | 1.5%, |
| Ve | 0.5%, |
| Synthetic wax SW3254 | 2%, |
| Synthetic wax M80 | 4%, |
| D5 | 10%, |
| Low viscosity silicone oil | 1%, |
| Silicone elastomer | 4%, |
| Glycerin | 5%, |
| Butylene glycol | 4%, |
| Glycerol polyether-26 | 5%, |
| Magnesium sulphate | 0.7%, |
| Pro-Xylane | 5%, |
| Water | 39.15%, |
| Modified starch (aluminum octyl succinate) | 0.5%, |
| Antiseptic (phenoxyethanol: 60+) | 0.3%, |
| Chlorphenesin: 280 | 0.15%, |
| Fragrance | 0.1%. |

The present example also relates to the aforementioned preparation method of the concealer sunscreen facial cream, including the following steps:
Step 1, weighing the raw materials according to the mass ratio of the main ingredients;
Step 2, the weighed raw materials are heated and dissolved, emulsified, cooled, and the finished product is obtained after the filling test is passed.

### Comparison Example 1

The method of comparison example 1 is basically the same as the method of example 1, except that Ve, aluminum octyl succinate and KF6038 are missing in the raw materials.

### Comparison Example 2

The method of comparison example 2 is basically the same as the method of example 1, except that KSP-300, KSP-100 and KP-545 are missing in the raw materials.

### Comparison Example 3

The method of comparison example 3 is basically the same as the method of example 1, except that Ve, KSP-100 and KP-545 are missing in the raw materials.

For 75 people with normal skin, 25 people in each group receive actual tests on the skin after use, and the results are shown in Table 1 below.

TABLE 1

| | | Comparison | Comparison | Comparison |
|---|---|---|---|---|
| Test Item | Example 1 | Example 1 | Example 2 | Example 3 |
| Concealer Factor | 5 | 2 | 3 | 1 |
| Sunscreen Time | 5 | 0 | 1 | 1 |
| Redness, Itching | No | Yes | Yes | Yes |
| Phenomenon | | | | |
| Moisturizing | 5 | 3 | 2 | 1 |
| Factor | | | | |
| Adverse Reaction | No | Yes | Yes | Yes |

In the above table 1, the higher the value of each index, the better the effect, from the above table data, it can be see that all the data in example 1 of the present invention are far better than those in comparison example 1-3; which indicates that the present invention uses phenoxyethanol as a preservative, with the effect of effectively inhibiting microorganisms in the air, which is not only non-toxic and harmless, but also achieves the effect of preservative, which is more natural and harmless and applicable to more people; the product is not a simple combination, and the scientific ratio and reasonable preparation method make it more effective, in addition, its scientific preparation method is suitable for batch production with higher production efficiency. The present invention adds KF6038 as a component, which interacts with polydimethylsiloxane PEG-10/15 cross-linked polymer/polydimethylsiloxane to finally realize the sunscreen function; KSP-300 and KSP-100 are also added to the present invention, which can realize excellent smoothness, elasticity and grease absorption; the invention also adds KP-545 to make the dispersivity of each component of the product stronger. The present invention adds Ve as a component, which interacts with other components to finally make the product has the effect of whitening and moisturizing and delaying skin aging. Synthetic wax SW3254 and synthetic wax M80 are added to the components of the present invention, which improves the emulsification of the whole system and makes each component give full play to its function; in addition, D5 is also added to provide a refreshing greasy skin feeling and reduce the greasy feeling.

The specific examples of the present invention are described above. It should be understood that the present invention is not limited to the above specific embodiments, and the person skilled in the art may make various variations or modifications within the scope of the claims, which does not affect the essence of the present invention.

1. A concealer sunscreen facial cream, characterized in that, comprising the following weight percentages of each component:

| | |
|---|---|
| polydimethylsiloxane PEG-10/15 cross-linked | 3%, |
| polymer/polydimethylsiloxane | |
| KF6038 | 1.5%, |
| polyglyceryl-3 diisostearate | 0.3%, |
| KSP-300 | 1.0%, |
| KSP-100 | 1.5%, |
| bismuth oxychloride | 1.8%, |
| coco-octanoic/capric acid | 2%, |
| ethylhexyl hydroxy stearate | 2%, |
| KP-545 | 2%, |
| isononyl isononanoate | 2%, |
| silicone oil | 1.5%, |
| Ve | 0.5%, |
| synthetic wax SW3254 | 2%, |
| synthetic wax M80 | 4%, |
| D5 | 10%, |
| low viscosity silicone oil | 1%, |
| silicone elastomer | 4%, |
| glycerin | 5%, |
| butylene glycol | 4%, |
| glycerol polyether-26 | 5%, |
| magnesium sulphate | 0.7%, |
| Pro-Xylane | 5%, |
| Water | 39.15%, |
| aluminum octyl succinate | 0.5%, |
| phenoxyethanol | 0.3%, |
| chlorphenesin | 0.15%, |
| fragrance | 0.1%. |

2. A preparation method of the concealer sunscreen facial cream according to claim 1, characterized in that, comprising the following steps:
Step 1, weighing the raw materials according to the mass ratio of the main ingredients;
Step 2, the weighed raw materials are heated and dissolved, emulsified, cooled, and the finished product is obtained after the filling test is passed.
